(19) United States
(12) Patent Application Publication
(10) Pub. No.: US 2025/0018126 A1
(43) Pub. Date: Jan. 16, 2025

(54) DRUG DELIVERY DEVICE AND DOSE RECORDING SYSTEM HEREWITH

(71) Applicant: Sanofi, PARIS (FR)

(72) Inventors: Tom Alexander Earwaker, Warwick, Warwickshire (GB); Oliver Charles Gazeley, Basel (CH); Adam Moyo Harvey-Cook, London (GB); Matthew Meredith Jones, Warwick, Warwickshire (GB); Harry Robert Lester, London (GB); David Aubrey Plumptre, Warwick, Warwickshire (GB); James Alexander Senior, Warwick, Warwickshire (GB); Robert Veasey, Warwick, Warwickshire (GB)

(21) Appl. No.: 18/712,184

(22) PCT Filed: Nov. 30, 2022
(86) PCT No.: PCT/EP22/83756

§ 371 Date: May 21, 2024

(30) Foreign Application Priority Data
Dec. 1, 2021 (EP) 21315266.3

(51) Int. Cl. A61M 5/315

(52) U.S. Cl. CPC A61M 5/3157; A61M 5/3158; A61M 2205/3306; A61M 2205/50

(57) ABSTRACT

The present disclosure refers to a drug delivery device for setting and dispensing doses of a liquid drug which may be used together with a module for detecting the amount of a dose selected and/or delivered. The device comprises a housing and a dose setting and drive mechanism with a dose setting member. The dose setting and drive mechanism is configured to perform a dose setting operation for selecting a dose to be delivered by rotating the dose setting member relative to the housing and a dose delivery operation for delivering the set dose by pushing the dose setting member axially towards the housing without relative rotation therebetween. The dose setting member comprises a set of axially directed stop teeth formed at its distal end which are configured to engage with a set of counterstop teeth located on the housing at the end of the dose delivery operation.

### CROSS-REFERENCE TO RELATED APPLICATIONS

The present application is the national stage entry of International Patent Application No. PCT/EP2022/083756, filed on Nov. 30, 2022, and claims priority to Application No. EP 21315266.3, filed on Dec. 1, 2021, the disclosures of which are incorporated herein by reference.

### TECHNICAL FIELD

The present disclosure is generally directed to a drug delivery device and to a dose recording system comprising a drug delivery device and an electronic module to be attached to the drug delivery device.

### BACKGROUND

Pen type drug delivery devices have application where regular injection by persons without formal medical training occurs. This may be increasingly common among patients having diabetes where self-treatment enables such patients to conduct effective management of their disease. In practice, such a drug delivery device may allow a user to individually select and dispense a number of user variable doses of a medicament or may be suitable for dispensing a fixed dose, e.g. in a push-pull-mechanism, or for choosing and dispensing preselected doses.

There are basically two types of drug delivery devices: resettable devices (i.e., reusable) and non-resettable (i.e., disposable). For example, disposable pen delivery devices are supplied as self-contained devices. Such self-contained devices do not have removable pre-filled cartridges. Rather, the pre-filled cartridges may not be removed and replaced from these devices without destroying the device itself. Consequently, such disposable devices need not have a resettable dose setting mechanism. Reusable devices need to have a resettable dose setting mechanism and a refillable medication container, e.g. a detachable cartridge holder provided for replacing a cartridge of medication contained therein when it is empty. The present disclosure is equally applicable for disposable devices, for example a device as disclosed in EP 2 890 434 B1, and in reusable devices, for example a device as disclosed in EP 2 890 435 B1. A drug delivery device indicating the axial end of dose position by a latching element is disclosed in WO 2010/052275 A2. WO 2021/099415 A1 discloses a drug delivery device with an electrical end of dose switch. Further drug delivery devices are also known from WO 2017/009102 A1, WO 2015/036346 A1 and WO 2016/001307 A1.

For such drug delivery devices the functionality of recording doses that are dialled and delivered from the pen may be of value to a wide variety of device users as a memory aid or to support detailed logging of dose history. Thus, drug delivery devices using electronics for such purposes are becoming increasingly popular in the pharmaceutical industry as well as for users or patients. For example, a dose recording system is known from WO 2021/116387 A1 comprising a drug delivery device and an electronic module which is removably mechanically coupled to the drug delivery device.

In known drug delivery devices, e.g. as disclosed in EP 2 890 434 B1, a dose setting and drive mechanism comprises a housing and a dose setting member wherein the mechanism is configured to perform a dose dialing operation for selecting a dose to be delivered by rotation of the dose setting member relative to the housing and a dose delivery operation for delivering the set dose by pushing the dose setting member axially towards the housing without relative rotation therebetween. The relative rotational position of these two parts at the end of the dose dialing operation is determined by a dial clicker detent. However, the dial clicker detent is deliberately designed to be overcome with relatively low torque and so does not provide a particularly accurate rotational datum. On the other hand, especially in drug delivery devices to be used together with an electronic module for detecting the doses delivered by the drug delivery device, it is desirable to have an accurate rotational datum for the 0 U dose dispensing stop condition, i.e. at the end of dose delivery and prior to setting a new dose.

### SUMMARY

It is an object of the present disclosure to provide improvements regarding a drug delivery device and a dose recording system comprising such a drug delivery device.

This object is solved for example by the subject matter defined in claim 1. Advantageous embodiments and refinements are subject to the dependent claims. However, it should be noted that the disclosure is not restricted to the subject matter defined in the appended claims. Rather, the disclosure may comprise improvements in addition or as an alternative to the ones defined in the independent claims as will become apparent from the following description.

One aspect of the disclosure relates to a drug delivery device for setting and dispensing doses of a liquid drug wherein the device comprising a dose setting and drive mechanism with a dose setting member and, e.g. a dose setting drum. The dose setting and drive mechanism is configured to perform a dose setting operation for setting, e.g. selecting or dialing, a dose to be delivered by the drug delivery device, for example by rotating the dose setting member and the dose setting drum relative to the housing, and a dose delivery operation for delivering the set dose, for example by rotating the dose setting drum relative to the dose setting member and the housing. In other words, the dose delivery operation may be effected by pushing the dose setting member (or a separate dispense button) axially towards the housing without relative rotation therebetween, that is during the dose delivery operation the dose setting member is rotationally stationary relative the housing. If the dose setting member comprises a set of axially directed stop teeth formed at its distal end which are configured to engage with a set of counterstop teeth located on the housing at the end of the dose delivery operation, an accurate rotational datum is provided at the end of the dose delivery operation by engagement of the stop teeth and the counterstop teeth. For example, the axially directed stop teeth may be formed on the outer rim of the dose setting member.

In other words, the dose setting member or a button component of the injection pen has a set of axially directed stop teeth at its proximal end (ie. end closest to the needle). These stop teeth may be formed on the proximal rim of a dose setting member sleeve and are configured to engage a set of counterstop teeth formed on an internal surface of the housing at the end of delivery of a dose.

The stop teeth may be formed on the dose setting member as portions with increased dose setting member wall thickness compared to adjacent portions with reduced dose setting member wall thickness. For example, the stop teeth may be configured as areas having substantially the same wall thickness as the main portion of the dose setting member, whereas the space between the stop teeth is defined by areas of reduced wall thickness. In addition or as an alternative, the stop teeth may have a proximally facing tooth root and a distally facing tooth tip, which may be slightly narrower compared with the circumferential width of the tooth root. Preferably, the dose setting member comprises a skirt, e.g. a substantially tubular sleeve, having a proximal end which permanently protrudes from the housing and a distal end which is permanently encased by the housing. In this example, the stop teeth may be formed at or near the distal end of the skirt, i.e. the stop teeth remain within the housing irrespective of the amount of dose set during the dose setting operation. This prevents that dirt or dust enters the interface formed by the stop teeth, thereby increasing the reliability of the device.

These stop teeth control and ‘true-up’ the rotational position of the dose setting member with respect to the housing and a dose setting drum at the end of each dose. This is important to ensure that clutch teeth between the dose setting member and the dose setting drum are aligned so that they reengage correctly when the dose setting member is released (allowing a subsequent dose to be dialled). Furthermore, it is important for accurate function of an electronic module that the rotational position of the dose setting member is consistent with respect to the dose setting drum at the end of each dose, if the module is intended to accurately detect the total relative rotational movement of the dose setting drum relative to the dose setting member to determine the size of the delivered dose. For example, 24 stop teeth may be provided equidistantly spaced at the distal end of the tubular sleeve of the dose setting member in a drug delivery device having 24 discrete dose increments per full rotation (360°) of the dose setting member.

Still further, if the dose setting member is pushed in with the pen in the 0 U dial condition, the engagement of these stop teeth and counterstop teeth prevent the user from rotating the dose setting member relative to the housing. Rotating the dose setting member in this condition could allow the user to manipulate the pen mechanism in a way that might advance or retract a piston rod, potentially leading to dose errors.

According to a further aspect of the disclosure, the dose setting member comprises two separate components permanently fixed to each other to function as a single component. For example, the dose setting member may comprise a tubular sleeve comprising the stop teeth and a ring of clutch teeth extending radially inwards, a ring of ramp teeth extending radially inwards and/or a profiling as a gripping surface and/or that the dose setting member comprises a button having a proximal end face and a stem extending distally from the end face, wherein the stem comprises at least one axially extending spline.

The dose setting operation for setting a dose to be delivered by the drug delivery device may include rotating or rotating and translating the dose setting member relative to the housing, e.g. along a helical path, whereas the dose delivery operation for delivering the set dose may include axially displacing the piston rod along an axis, preferably the central longitudinal axis of the piston rod. Optionally, the dose delivery operation for delivering the set dose may include moving the dose setting member relative to the housing, e.g. axially displacing the dose setting member.

The drug delivery device may be suitable for setting and dispensing variable doses of a liquid drug allowing a user to individually select and dispense a number of user variable doses of a medicament. As an alternative, the drug delivery device may be a fixed dose device, e.g. a push-pull-device permitting only dispensing a pre-defined fixed dose. As a further alternative, the drug delivery device may be suitable to select between one or more preset doses, e.g. doses exceeding or falling below a threshold as disclosed in WO 2016/128424 A1. Thus, the terms “dose setting”, “dose selecting” and “dose dialing” are used herein not limiting the drug delivery device to a certain mode of operation.

The dose setting drum may only display the currently set dose without causing the dose selection and/or dose delivery. The dose setting drum may move, e.g. rotate, together with, for example, the dose setting member relative to the housing during at least one of the dose setting operation and the dose delivery operation. In an example, the dose setting drum rotates on a helical path during dose setting and during dose delivery.

The dose setting and drive mechanism of the drug delivery device may comprise a dial clicker which defines discrete positions for the dose setting member with respect to the housing when dialling. The dial clicker may be formed by a flexible clicker arm and a ring of axially extending splines. In more detail, the dose setting and drive mechanism may comprise a drive sleeve permanently rotationally fixed to the dose setting member, and e.g. axially displaceable relative to the dose setting member, and an inner housing insert permanently rotationally, and e.g. axially, fixed to the housing, wherein the dial clicker is formed by a flexible clicker arm of the drive sleeve and a ring of axially extending splines formed on the inner housing insert. During dose setting, the drive sleeve is rotated relative to the inner housing and the clicker arm bounces over the splines of the inner housing insert, thereby generating clicking noises. During dose delivery, the drive sleeve (including the flexible clicker arm) moves axially without rotation. Therefore, no clicking noise is generated by the dial clicker during dose delivery.

In an example of the present disclosure, the dose setting drum comprises a set of clutch teeth that are engagable with a mating set of teeth on the dose setting member. Thus, the clutch teeth and the mating teeth may be engaged during a dose setting, e.g. dialing, operation to prevent rotation therebetween whereas the clutch teeth and the mating teeth may be disengageable during a dose delivery operation allowing relative rotation between the dose setting drum and the dose setting member. One aspect of the present disclosure is directed to an arrangement of the clutch clutch teeth on an external surface of the dose setting drum.

If the clutch teeth project radially from the external diameter of the dose setting drum, they interact with the mating teeth formed on the inner diameter of the dose setting member to transfer torque at the largest possible diameter. This means that they are stronger and more resistant to torque applied by the user, for example in a dial stop condition, than would be the case if they were formed on a smaller diameter. Forming the clutch teeth on the external diameter of the dose setting drum also simplifies molding tool construction, as it allows the thread to run along the entirety of the inner cylindrical surface of the part to be unscrewed from the core pin without the need to first disengage the tool from the clutch teeth.

According to an example of the present disclosure, the clutch teeth are equidistantly spaced around the circumference of the dose setting drum. In more detail, the number of clutch teeth formed on the dose setting drum may be a whole fraction of the number of rotational positions in which these clutch teeth are engageable with the mating teeth of the dose setting member. For example, the number of clutch teeth formed on the dose setting drum may be equal to half the number of rotational positions in which these clutch teeth are engageable with the mating teeth of the dose setting member.

Preferably, the drug delivery device, e.g. a pen type injector, is suitable for use with an attachable electronic module configured to capture information relating to the size of doses delivered. The module may be detachable from the drug delivery device and/or may be fixed after attachment to the drug delivery device.

For use with an electronic module detecting the amount of dose delivered by the drug delivery device, the dose setting drum may comprise an array of, preferably equidistantly spaced, encoder features formed in a circular pattern at its proximal end for detecting rotational movement of the dose setting drum relative to the dose setting member. Further, the dose setting member may have at least one aperture located in a proximal end surface that is coincident, i.e. overlapping, with at least a portion of the array of encoder features of the dose setting drum. According to the present disclosure, the aperture and encoder features are coincident, if during a full rotation (360°) of the encoder features, each encoder feature passes the aperture one time. With the aperture being provided in a proximal end surface, the encoder features are preferably facing proximally. For example, two apertures are formed, e.g. at 180° opposite positions, in the proximal end surface of the dose setting member. Detecting rotation of the dose setting drum relative to the dose setting member is facilitated if the at least one aperture spans at least one, or more preferably two or more of the encoder features of the dose setting drum. The at least one aperture may be located at the distal end of a groove formed in the proximal end surface of the dose setting member. For example two concentric grooves may be formed in the proximal end surface of the dose setting member. The at least one aperture is preferably located at the distal end of the outer groove, whereas the inner groove may constitute a mechanical coding.

According to an aspect of the present disclosure, the clutch teeth provided at the distal end of the dose setting drum may have the additional function of the encoder features. In other words, the relative rotational movement is detectable by a sensor of an electronic module to determine the size of the dose that is delivered. However, the present disclosure is not limited to the encoder features being the clutch teeth. Rather, the encoder features but could simply be flags for an optical encoder system.

The dose setting member may be provided with attachment features for releasably mounting a separate detachable module to the drug delivery device, e.g. an electronic module. In more detail, the dose setting member may be provided with at least one, e.g. substantially circular, groove extending in a proximal end face of the dose setting member with the attachment features being provided in or adjacent to the groove. The dose setting member may be provided with two concentric grooves. In an example, the attachment features may comprise at least one, e.g. two opposite located, radial aperture(s) provided in the dose setting member. The aperture(s) may be provided facing radially outwards from a groove, e.g. from the outer groove of two concentric groves.

In an example, the maximum external diameter of the dose setting member is smaller than the external diameter of the housing. In more detail, the maximum external diameter of the dose setting member may be smaller than or equal to the internal diameter of a region of the housing located adjacent to the dose setting member.

The electronic module may use a set of optical IR emitters/detectors to encode doses delivered from the drug delivery device, e.g. a variable dose pen. In order to reduce the level of stray IR radiation, for example from high ambient light levels if the pen is used outdoors, that may interfere with correct performance of the module sensors, the dose setting member components of the pen may have features that are intended to reduce/suppress levels of reflected IR radiation. In particular, the module components and/or the dose setting member may contain an IR absorbing masterbatch and/or may have a spark-eroded textured surface finish.

A dose recording system according to the present disclosure may comprise a drug delivery device as described herein and an electronic module as described herein for releasable or fixed attachment to the dose setting member of the drug delivery device. The module may also be integrated into the dose setting member. For example, the module may comprise a sensor, a processor configured to control operation of the at least one sensor and to process and/or store signals from the at least one sensor, and attachment features for attaching the module to the drug delivery device. The module may comprise a cap for receiving a proximal end of the dose setting member.

The electronic module may comprise a rotation sensor for detecting rotation of a sensed element which is rotationally fixed to a component of the drug delivery device, wherein the sensed element rotates during dose setting and/or during dose delivery relative to the dose setting member to which the electronic module is attached. The sensed element may be fixed to the dose setting drum. It may be an encoder ring with a pattern (e.g. an optical pattern or a magnetic pattern) that can be detected by the rotation sensor. The rotation sensor may comprise at least one optical sensor and/or at least one magnetic sensor.

According to a separate aspect of the present disclosure, the sensor of the module comprises at least one set of optical IR emitters/detectors that can be located coincident, i.e. overlapping, with the at least one aperture in a proximal end surface of the dose setting member. In more detail, the module may comprise a chassis holding the sensor and the processor wherein the chassis comprises at least one light pipe extending through the at least one aperture in the proximal end surface of the dose setting member. This results in a highly reliable and precise detection of the doses delivered using an optical sensor unit which is located spaced from encoder features. The chassis may include features as disclosed in WO2021/214275.

Alternatively or additionally to at least one optical sensor, the electronic module may comprise one or more mechanically actuated switches, or at least one magnetic sensor provided for detecting the doses delivered by the drug delivery device.

The maximum external diameter of the dose setting member may be equal to or smaller than the inner diameter of the cap, thereby permitting to attach the module on the dose setting member. The maximum external diameter of the module may be equal to, similar to, or slightly larger than the external diameter of the housing, e.g. exceeding the external diameter of the housing by about 0 mm to about 5 mm. This configuration does not hinder the dose delivery operation, even if the dose setting member has to spin when the dose is delivered. In addition, the risk of the module being inadvertently dislodged or damaged is minimized and inadvertently applying excessive torque due to a too large dose setting member diameter is prevented.

The electronic module of the dose recording system may be a re-usable module for releasable attachment to a drug delivery device. The module may comprise an outer cap with a central axis, a chassis which is at least partially retained within the cap and a PCB or PCBA comprising the memory and the processor. For example, the PCBA and the electrical power supply may be retained in the cap and the chassis. Further, the light source(s) and the optical sensor(s) may be arranged on a circular region about the central axis, with the first light source and the first optical sensor being angularly offset from the second light source and the second optical sensor. In addition, a light guide may be provided in the chassis guiding light from an internal light source to an external surface of the module in order to display information regarding the status or operation mode of the module.

The module may comprise attachment features for releasably or fixedly mounting the module to the proximal end of the drug delivery device, e.g. to the dose setting member, to a button or to the housing. For example, the attachment features may comprise at least one flexible clip for axially and rotationally fixing the module to the dose setting member. The at least one flexible clip may be received within the cap and may extend substantially distally from a chassis holding the sensor and the processor.

According to a further aspect of the present disclosure, the dose setting member may comprise a mechanical coding and the module may comprise a mechanical counter-coding which engages with the mechanical coding when the module is attached to the drug delivery device. This may prevent attaching a non-matching module to a drug delivery device. In addition, torque may be transmitted from the module to the dose setting member via the mechanical coding and the mechanical counter-coding.

The electronic module of the dose recording system may comprise an electronic system for use with a drug delivery device suitable for recording doses that are delivered from the drug delivery device. The electronic system may comprise an electrical power supply, e.g. a battery, like a coin cell type battery, a memory for storing data, a processor configured to control operation of the electronic system and coupled to the electrical power supply and to the memory. In addition, the electronic system may comprise at least one, preferably two, optical sensor unit(s), e.g. a first light source with a corresponding first optical sensor and a second light source with a corresponding second optical sensor, which are in communication with the processor. The optical sensors may be suitable for detecting a movement of an encoder of the drug delivery device, especially segments with different reflectivity, e.g. of the dosing sleeve, wherein the movement is indicative of doses that are dialed (i.e. selected) and/or delivered from the drug delivery device. There are several different ways suitable to implement the optical sensor unit(s). For example, the optical sensor unit(s) may comprise a radiation detector comprising an electromagnetic radiation emitter, e.g. an LED, like an IR-LED, e.g. an NIR-LED, and a radiation detector.

In one example, the encoder and the optical sensor units are in a quadrature arrangement, i.e. they are a quarter wave out of phase, which means that if both light sources simultaneously emit light, only one sensor changes state for each unit dispensed. For example, this is achieved by providing two optical sensors circumferentially offset by n*30°+15° with n being an integer number. As the encoder and the sensor units are moved relative to each other, one of the optical sensors which previously received the light now does not receive the emitted light or vice versa. This may be achieved by the encoder selectively reflecting light. For example, a ring of teeth may be provided such that the teeth reflect light whereas the free space between adjacent teeth does not reflect light or reflects less light. As an alternative, areas reflecting light and areas absorbing light may be alternately provided. As a further alternative, the encoder may selectively block light. In other examples the encoder and the optical sensor units may be in anti-phase arrangement. In a still further alternative, the encoder and the optical sensor units are not in an anti-phase arrangement, such that if both light sources simultaneously emit light, none or only one or all optical sensors detect the light depending on the relative position of the encoder. The encoder may work as described in WO2019/101962A1.

The electronic module of the dose recording system may be configured as a re-usable or fixedly attachable clip-on module for an injection device. As an alternative, the electronic system may be a unit or module integrated (built in) into an injection device. The terms electronic system and (electronic) module are used in the following for both alternatives. The functionality of recording doses may be of value to a wide variety of device users as a memory aid or to support detailed logging of dose history. It is envisaged that the electronic system, e.g. an electronic module, could be configured to be connectable to a mobile phone, or similar, to enable the dose history to be downloaded from the system on a periodic basis.

The electronic dose recording system may further comprise a communication unit for communicating with another device. Preferably, the electronic module of the dose recording system is configured such that it may be switched from a first state having lower energy consumption into the second state having higher energy consumption, thereby inducing the communication unit to establish said communication with another device, e.g. a synchronization or pairing operation. An electronic control unit may issue a command, e.g. a signal, to another unit of the electronic dose recording system such that this unit is switched on or rendered operational. This unit may be the communication unit for communicating with another device, e.g. a wireless communications interface for communicating with another device via a wireless network such as Wi-Fi or Bluetooth, or even an interface for a wired communications link, such as a socket for receiving a Universal Series Bus (USB), mini-USB or micro-USB connector. Preferably, the electronic dose recording system comprises an RF, Wi-Fi and/or Bluetooth unit as the communication unit. The communication unit may be provided as a communication interface between the dose recording system or the drug delivery device and the exterior, such as other electronic devices, e.g. mobile phones, personal computers, laptops and so on. For example, dose data may be transmitted by the communication unit to the external device. The dose data may be used for a dose log or dose history established in the external device.

According to a still further aspect of the present disclosure, the electronic dose recording system further has a sleeping state in which the light sources are not activated (not provided with power from the power source). The electronic dose recording system may further comprise at least one switch and/or a motion sensor suitable for detecting movement of the electronic system. In this example, the processor may be configured to maintain the sleeping state if no switch activation or movement is detected by the at least one motion sensor and to switch into the first low-power-consumption state or into the at least one further state if a switch activation or a movement is detected by the at least one motion sensor. Generally, a sleeping state or mode may be a mode in which all functionalities of the module are at minimal or virtually zero power consumption but which does not require a system boot up in the event that the electronic system (or the drug delivery device) is taken out of sleeping mode.

The present disclosure further pertains to a drug delivery device with the electronic system as described above which drug delivery device may comprise a container, e.g. a cartridge, containing a medicament.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (SIRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly (A21), Arg (B31), Arg (B32) human insulin (insulin glargine); Lys (B3), Glu (B29) human insulin (insulin glulisine); Lys (B28), Pro (B29) human insulin (insulin lispro); Asp (B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, for example, B29-N-myristoyl-des (B30) human insulin, Lys (B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N-(N-lithocholyl-gamma-glutamyl)-des (B30) human insulin; B29-N-(ω-carboxyheptadecanoyl)-des (B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 MAR701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014 (E). As described in ISO 11608-1:2014 (E), needle-based injection systems may be broadly distinguished into multi-dose container systems and single-dose (with partial or full evacuation) container systems. The container may be a replaceable container or an integrated non-replaceable container.

As further described in ISO 11608-1:2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014 (E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014 (E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

The terms “axial”, “radial”, or “circumferential” as used herein may be used with respect to a main longitudinal axis of the device, the cartridge, the housing or the cartridge holder, e.g. the axis which extends through the proximal and distal ends of the cartridge, the cartridge holder or the drug delivery device.

A non-limiting, exemplary embodiment of the disclosure will now be described with reference to the accompanying drawings, in which:

### BRIEF DESCRIPTION OF THE FIGURES

FIG. 1 shows an embodiment of a drug delivery device in a perspective view;

FIG. 2 shows the component parts of the device of FIG. 1;

FIG. 3a shows a sectional view of the device of FIG. 1 together with an electronic module;

FIG. 3b shows a sectional view of the device of FIG. 1 together with attached electronic module;

FIG. 4 shows a view on the proximal end of a dose setting member button of the device of FIG. 1;

FIG. 5 shows a view on the proximal end of the device of FIG. 1;

FIG. 6 shows a sectional view of the proximal end of the device of FIG. 1;

FIG. 7 shows a dose setting drum of the device of FIG. 1 with an enlarged detail;

FIG. 8 shows a housing of the device of FIG. 1;

FIG. 9 shows a dose setting member sleeve of the device of FIG. 1;

FIG. 10 shows the proximal end of the dose setting member sleeve of FIG. 9; and

FIG. 11 shows the proximal end of the dose setting drum of FIG. 7 with the dose setting member sleeve of FIG. 9.

### DETAILED DESCRIPTION

In the figures, identical elements, identically acting elements or elements of the same kind may be provided with the same reference numerals.

In the following, some embodiments will be described with reference to an insulin injection device. The present disclosure is however not limited to such application and may equally well be deployed with injection devices that are configured to eject other medicaments or drug delivery devices in general, preferably pen-type devices and/or injection devices.

Embodiments are provided in relation to injection devices, in particular to variable dose injection devices, which record and/or track data on doses delivered thereby. This data may include the size of the selected dose and/or the size of the actually delivered dose, the time and date of administration, the duration of the administration and the like. Features described herein include the arrangement of sensing elements and power management techniques (e.g. to facilitate small batteries and/or to enable efficient power usage).

The drug delivery device 1 shown in the Figures is based on the same general working principle as the disposable injection pen disclosed in EP 2 890 434 B1 to which reference is made regarding the main functions and the operation modes. However, the embodiment depicted in the Figures is improved in some aspects as described below in more detail and is adapted for use with a detachable electronic module 2. Non-limiting example of such a module are disclosed in WO 2021/116387 A1 and PCT/EP2021/060631 to which reference is made regarding the main functions and the operation modes of an electronic module.

Although described with reference to a disposable injection pen similar to the one disclosed in EP 2 890 434 B1, the present disclosure is applicable to other disposable or reusable drug delivery device, including but not limited to the injection devices disclosed in one of WO 2004/078239 A1, WO 2014/033195 A1, WO 2009/132777 A1, WO2005/018721, U.S. Pat. Nos. 5,693,027, 6,663,602 or 7,241,278.

“Distal” is used herein to specify directions, ends or surfaces which are arranged or are to be arranged to face or point towards a dispensing end of the drug delivery device or components thereof and/or point away from, are to be arranged to face away from or face away from the proximal end. On the other hand, “proximal” is used to specify directions, ends or surfaces which are arranged or are to be arranged to face away from or point away from the dispensing end and/or from the distal end of the drug delivery device or components thereof. The distal end may be the end closest to the dispensing and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end where a needle unit is or is to be mounted to the device, for example.

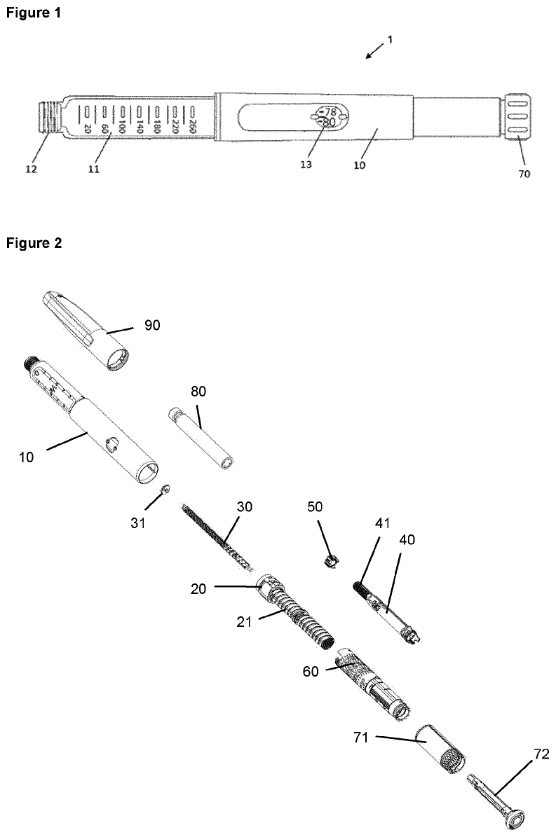

FIGS. 1 and 2 depict a medicament delivery device or drug delivery device 1 comprising an outer housing 10, an inner housing insert 20, a piston rod 30, a drive sleeve 40, a nut 50, a dose setting drum 60, a dose setting member 70, a cartridge 80 and a cap 90. A needle arrangement (not shown) comprising a needle hub and e.g. a needle cover may be provided as additional components.

The outer housing 10 is a generally tubular element having a distal part, which forms a cartridge holder 11 for receiving cartridge 80 and which is provided with a thread 12 or the like for attaching a needle hub, and a proximal part, which encases component parts of a dose setting and drive mechanism. In a preferred embodiment, the outer housing 10 is transparent, with the proximal part being optionally provided with an opaque layer. The housing 10 comprises a transparent window 13. A sectional view of housing 10 is depicted in FIG. 8 in which a series of counterstop teeth 14 is visible formed on an inner surface of housing 10.

The inner housing insert 20 is an inner body with a generally tubular configuration having different diameter regions. The inner housing insert 20 is received in the proximal part of housing 10 and is permanently fixed therein to prevent any relative movement of the inner housing insert 20 with respect to the housing 10. An external thread 21 is provided on the outer surface of the inner housing insert 20. Further, splines are provided on the inner surface of the inner housing insert 20 and its distal end is provided with an inner thread.

The piston rod 30 is an elongate element having two external threads with different leads, preferably of opposite hand which overlap each other. One of these threads engages the inner thread of the inner housing insert 20. A disk-like bearing 31 may be attached at the distal end of the piston rod 30 to allow relative rotation between the bearing 31 and the piston rod 30. As an alternative the bearing may be attached to the piston rod 30 as a one-piece component via a predetermined breaking point.

The drive sleeve 40 is a generally tubular element having different diameter regions. A distal region of the drive sleeve 40 has an external thread 41. An inner surface of the drive sleeve 40 has an inner thread engaging one of the external threads of the piston rod 30. The drive sleeve 40 surrounds the piston rod 30 and is at least partly located within inner housing insert 20. The drive sleeve 40 further has a flexible clicker arm formed by a U-shaped opening in the skirt of the drive sleeve 40. The clicker arm engages the internal splines in the inner housing insert 20 and is allowed to flex radially inwards during relative rotation between the drive sleeve 40 and the inner housing insert 20. This radial deflection is permitted as long as the clicker arm is in alignment with a pocket in the dose setting member 70, but may be blocked if the clicker arm is not in alignment with a pocket in the dose setting member 70, thereby preventing relative rotation between the drive sleeve 40 and the inner housing insert 20. At its proximal end, the drive sleeve 40 may comprise a spring arm abutting the dose setting member 70 and biasing the drive sleeve 40 distally with respect to the dose setting member 70. The internal surface of the drive sleeve 40 is provided with at least one axially extending groove permanently engaging splines on the dose setting member 70 such that any rotation of the dose setting member 70 is transferred to the drive sleeve 40.

The nut 50 is provided between the inner housing insert 20 and the drive sleeve 40. External ribs of the nut 50 engage the internal splines of the inner housing insert 20. An internal thread of the nut 50 engages the external thread 41 of the drive sleeve 40. As an alternative, splines and ribs could be provided on the interface between the nut 50 and the drive sleeve 40 and threads could be provided on the interface between the nut 50 and the inner housing insert 20. As a further alternative, the nut 50 may be designed as e.g. a half nut. Further, at least one, e.g. four, rotational hard stops are provided on nut 50 for interaction with corresponding stops on the drive sleeve 40 at the proximal end of thread 41.

The dose setting drum 60 is a generally tubular element with an internal thread engaging the external thread 21 of the inner housing insert 20. Thus, the dose setting drum 60 is interposed between the inner housing insert 20 and the housing 10. A series of numbers is provided, e.g. printed, on the outer surface of the dose setting drum 60 which forms a display member. The numbers are arranged on a helical line such that only one number or only a few numbers are visible in through window 13 of the housing 10. As will be explained in more detail below, the dose setting drum 60 is provided with a ring of clutch teeth 61 near its proximal end (FIG. 7) for engaging corresponding clutch teeth 73 of the dose setting member 70. Further, an optional clicker arm 62 may be provided, e.g. near the distal end of dose setting drum 60, for engaging a ratchet profile 74 of the dose setting member 70.

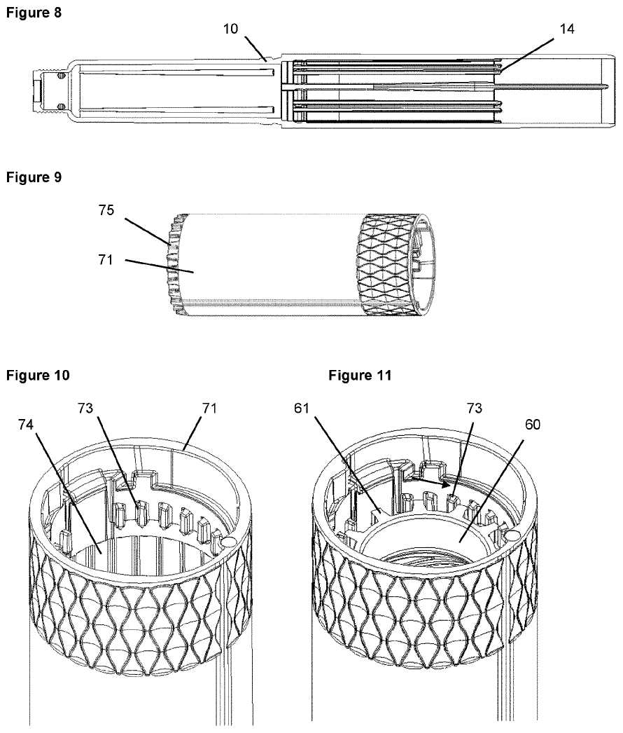

The dose setting member 70 comprises two separate components, namely a tubular sleeve 71 (FIG. 9) and a button 72, permanently fixed to each other to function as a single component. The tubular sleeve 71 of dose setting member 70 comprises an internal ring of clutch teeth 73 near its proximal end, an internal ring of ramped teeth forming the ratchet profile 74 and stop teeth 75 at its distal end for engaging counterstop teeth 14 of the housing 10. The button 72 comprises a proximal end face and a stem extending distally from this end face. As shown in FIG. 4, two radial apertures 76 and two axial apertures 77 both formed in an outer groove 78 which is concentrically disposed with respect to an inner groove 79 formed in the end face of button 72 of dose setting member 70. The stem of button 72 is provided with the pocket into which the clicker arm of the drive sleeve 40 may flex and comprises at least one axially extending spline permanently engaging a respective groove formed in the drive sleeve 40.

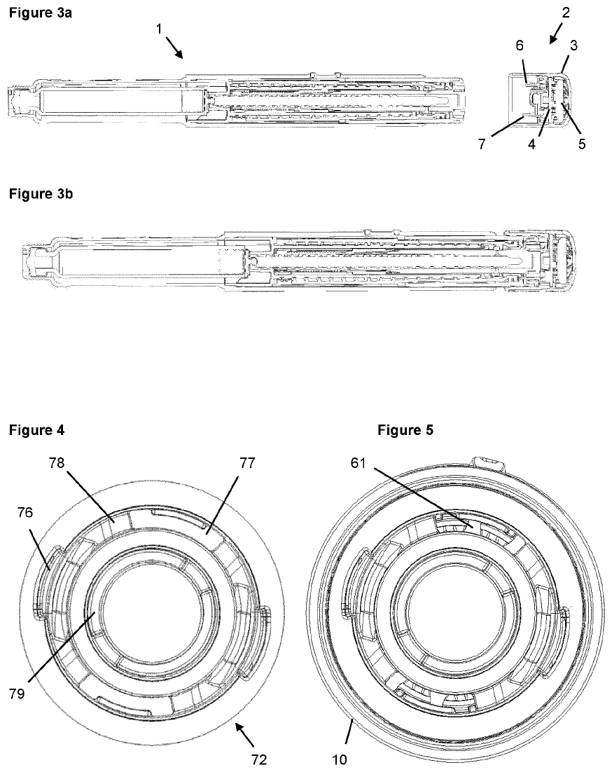

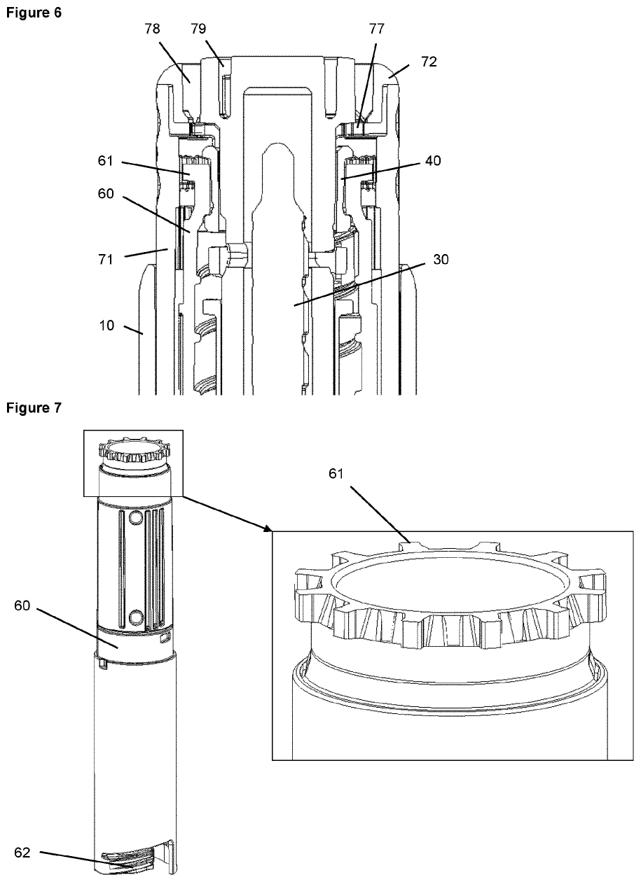

As shown in FIGS. 3a and 6, the tubular sleeve 71 of dose setting member 70 may be provided with a profiled proximal region facilitating gripping and rotating the dose setting member 70. The maximum external diameter of the dose setting member 70, especially of the tubular sleeve 71, is smaller than the external diameter of the housing 10. In more detail, the maximum external diameter of the dose setting member 70 is smaller than the internal diameter of a region of the housing 10 located adjacent to the dose setting member 70.

The cartridge 80 includes a pre-filled, necked-down cartridge reservoir, which may be typically made of glass. A rubber type bung or stopper is located at the proximal end of the cartridge reservoir, and a pierceable rubber seal is located at the other, distal, end. A crimped annular metal band is used to hold the rubber seal in place. The cartridge 80 is provided within the cartridge holder 11 with bearing 31 of piston rod 30 abutting the bung.

FIG. 1 shows the device 1 without cap 90. The cap 90 may be attached to the distal end of the device 1, thus covering the cartridge holder 11. The cap 90 may be releasably snapped onto the housing 10 and can be taken off for use of the device 1.

The electronic module 2 may be releasably attached or fixed to the proximal end, e.g. to the dose setting member 70, of the drug delivery device 1, thereby forming a dose recording system. The module 2 may comprise a cup-shaped outer cap 3 retaining a PCBA 4 with a processor and a battery 5. The module 2 further comprises a sensor arrangement that is connected to the processor and operable to generate measurement data indicative of the dose setting operation and/or the dose delivery operation. For this purpose the sensor arrangement comprises at least one LED and one or more photo detector(s) together forming an optical sensor. Alternative sensor types could be implemented in addition to the LED and the photo detector or as an alternative thereto. Such alternative sensor types may include but are not limited to optical sensors, acoustic sensors, capacitive sensors, electrical switches. The PCBA 4 may further comprise or may be connected to a communication unit comprising e.g. a wireless Bluetooth® communication interface connected to the processor and operable to establish communication with another (external) device, e.g. a smartphone. The communication unit is operable to transfer data, e.g. measurement data, to said other device. Still further, an electronic user feedback generator may be connected to the processor and operable to generate a feedback signal to a user. In an exemplary arrangement, the electronic user feedback generator comprises an LED for generating optical feedback signals. In addition to the LED or as an alternative the electronic user feedback generator may comprise a sounder and/or a vibration motor.

The module 2 further comprises a chassis which is in snap-fit engagement with the cap 3, thereby axially and rotationally constraining the cap and the chassis. The chassis is transparent or translucent and may be made from Polycarbonate or the like. The chassis receives the PCB unit 4. Further, the chassis comprises two light pipes 6 which are axially extending protrusions of the chassis with polished side walls and diffuse end faces forming a sensor side end and an encoder side end. In other words, light may enter or leave the light pipes at the opposite sensor side end and encoder side end but is guided (reflected) by the side walls. In an example the light pipes 6 may be rotationally offset by 45° with respect to each other. The light pipes 6 are arranged to fit into or extend through one of the apertures 77 in button 72 when the module 2 is attached to the drug delivery device 1. For this purpose, the chassis further comprises attachment features in the form of two flexible clips 7 rotationally offset by e.g. 180° for engaging radial apertures 76 in button 72.

The dose setting and drive mechanism of the drug delivery device is configured to perform a dose setting or dialing operation for selecting a dose to be delivered by the drug delivery device and a dose delivery operation for delivering the set dose.

During the dose setting operation, clutch teeth 61 of the dose setting drum 60 engage clutch teeth 73 of the dose setting member 70. Thus, the dose setting drum 60, the dose setting member 70 and the drive sleeve 40 (which is splined to the dose setting member 70) are rotationally coupled. A user may set a dose by rotating tubular sleeve 71 of dose setting member 70 such that the dose setting drum 60, the dose setting member 70 and the drive sleeve 40 wind out of housing 10 along a helical path defined by thread 21 of the inner housing insert 20 guiding the thread of dose setting drum 60. The pitch of the threaded interface between the piston rod 30 and the drive sleeve 40 corresponds to the pitch of the threaded interface between the inner housing insert 20 and the dose setting drum 60 such that the piston rod 30 remains stationary held by its threaded interface with the inner housing insert 20. The selected dose amount is visible through window 13 in the housing 10. During this rotation of the dose setting drum 60, the dose setting member 70 and the drive sleeve 40 relative to the housing, the flexible clicker arm of drive sleeve 40 snaps over the internal axial splines of the inner housing insert 20 thereby defining discrete rotational positions. In the depicted exemplary embodiment, one full rotation of the dose setting member corresponds to 24 dose increments. For example, where the injection device 1 is configured to administer human insulin, the dosage increments may be displayed in so-called International Units (IU), wherein one IU is the biological equivalent of about 45.5 micrograms of pure crystalline insulin (1/22 mg).

At the maximum settable dose of, e.g. 80, units stop features may engage to prevent further setting or dialing. Further, during dose setting, the drive sleeve 40 rotates relative to the inner housing insert 20 such that the nut 50 which is rotationally fixed to the inner housing insert 20 via axially extending ribs and splines is moved on thread 41 of the drive sleeve 40. The last dose nut 50 provides the function of counting the number of dispensed units. The nut 50 locks the device 1 at the end of life and as such no more drug can be dialed or dispensed by the user. Rotation of the drive sleeve 40 during setting, e.g. dialing, causes the nut 50 to advance along thread 41. The nut 50 is free to slide axially within the inner housing insert 20 at all times which allows advancement of the nut 50. At the end of life condition, the stop features of the last dose nut 50 contact the corresponding features on the drive sleeve 40. The splined contact with inner housing insert 20 reacts any torque transmitted by these stop features 47.

With the desired dose dialed, the device 1 is ready for dose dispensing. This basically requires pushing dose setting member 70 which will result in a disengagement of the clutch teeth 61, 73. As mentioned above, when setting a dose the dose setting member 70 is ‘biased out’ by the flexible arm of drive sleeve 40 and the clutch teeth 61, 73 which rotationally lock the drive sleeve 40, dose setting member 70 and dose setting drum 60 together are engaged. Upon pressing the dose setting member 70 the clutch teeth 61, 73 disengage and relative rotation between the dose setting drum 60 and the dose setting member 70 is possible. In all conditions the drive sleeve 40 and the dose setting member 70 remain rotationally locked. Thus, with the clutch disengaged (dose setting member 70 pushed in) dose setting member 70 and drive sleeve 40 are rotationally locked together while the dose setting member 70, the drive sleeve 40 and the dose setting drum 60 are still axially coupled. At the same time the relative axial movement of the dose setting member 70 with respect to the drive sleeve 40 results in the pocket on the stem being shifted relative to the flexible clicker arm. Thus, the flexible clicker arm is prevented from flexing inwards. This activation of the locking feature prevents the flexible clicker arm from overcoming splines of the inner housing insert 20 if the dose setting member 70 is pressed. In this condition, the drive sleeve 40 and the dose setting member 70 are rotationally constrained to the inner housing insert 20 thus preventing any rotation relative to the housing 10 during delivery of the dose.

With the desired dose dialed the dose setting member 70 can be depressed and the piston rod 30 is driven forward to dispense drug from the cartridge. The interaction of mating threads between the piston rod 30, drive sleeve 40 and inner housing insert 20 delivers a mechanical advantage. During dose dispensing a dispense clicker is active which involves the ratchet profile 74 of the dose setting member 70 and the clicker arm 62 of dose setting drum 60. The dispense clicker provides primarily audible feedback to the user that drug is being dispensed. Relative rotation is only allowed in one direction. During dose delivery, the drive sleeve 40 does not rotate relative to the inner housing insert 20. Thus, the rotational position of nut 50 is not changed during dose delivery and the nut 50 remains at its position on the drive sleeve 40 set during dose setting or dialing.

The dose setting member 70, more specifically the tubular sleeve 71, has a set of axially directed stop teeth 75 at its distal end. These teeth 75 are formed on the proximal rim of the tubular sleeve 71 and are configured to engage a set of counterstop teeth 14 formed on an internal surface of the housing 10 at the end of delivery of a dose. In the depicted example, 24 stop teeth 75 are provided at the distal end of the tubular sleeve 71. As can be seen in FIG. 9, the space between the stop teeth 75 is not fully open in the radial direction, but has a reduced wall thickness. In other words, the stop teeth 75 are formed as portions with increased wall thickness compared to adjacent portions with reduced wall thickness. In contrast to a saw-tooth shape of stop teeth known from other drug delivery devices, the stop teeth 75 may be symmetrically formed in circumferential direction as depicted in FIG. 9.

These teeth 14, 75 control and ‘true-up’ the rotational position of the dose setting member 70 with respect to the housing 10 and the dose setting drum 60 at the end of each dose. This is important to ensure that the clutch teeth 61, 73 between the dose setting member 70 and the dose setting drum 60 are aligned so that they reengage correctly when the dose setting member 70 is released (allowing a subsequent dose to be dialed). Furthermore, it is important for accurate function of the module 2 that the rotational position of the dose setting member 70 is consistent with respect to the dose setting drum 60 at the end of each dose. This is because the module 2 is intended to accurately detect the total relative rotational movement of the clutch teeth 61 on the dose setting drum 60 relative to the dose setting member 70 to determine the size of the delivered dose. Without the above mentioned teeth 14, 75 between the dose setting member 70 and housing 10, the relative rotational position of these two parts at the end of a dose would only be determined by the dial clicker (flexible clicker arm of drive sleeve 40 and axial splines of inner housing insert 20). However, as the dial clicker is deliberately designed to be overcome with relatively low torque, it does not provide a particularly accurate rotational datum. Still further, if the dose setting member 70 is pushed in with the pen 1 in the 0 U dial condition the engagement of these teeth 14, 75 prevent the user from rotating the dose setting member 70 relative to the housing 10. Rotating the dose setting member 70 in this condition would allow the user to manipulate the pen mechanism in a way that might advance or retract the piston rod 30, potentially leading to dose errors.

The drug delivery device 1 is suitable to be used with or without module 2. If module 2 is fitted over dose setting member 70, flexible clips 7 engage and snap into radial apertures 76 of button 72, while light pipes 6 extend into axial apertures 78. When used in conjunction with module 2, the ring of clutch teeth 61 of the dose setting drum 60 has the additional function of an encoder.

These radially outwardly projecting clutch teeth 61 located on the outer diameter of the dose setting drum 60 may be detected by the optical sensor. As shown in FIG. 7, in the exemplary embodiment, there are only twelve equidistantly spaced clutch teeth 61, yet they combine with a set of twenty-four mating teeth 73 on the dose setting member 70 to provide twenty-four discrete engagement locations (thus allowing 24 selectable dose increments per revolution of the dose setting member when dialing and delivering doses). This arrangement has a number of advantages. Because the clutch teeth 61 project radially from the external diameter of the dose setting drum 60, they interact with the teeth 73 formed on the inner diameter of the dose setting member 70 to transfer torque at the largest possible diameter. This means that they are stronger and more resistant to torque applied by the user. A smaller number of wide teeth (i.e. 12 ‘double width’ teeth rather 24 single width teeth) also provides a stronger means of transferring torque from the dose setting drum 60 to the dose setting member 70. A smaller number of wide teeth 61 also provides a much better target for the optical detector of the module 2 because the individual reflective/non-reflective regions are wider and therefore provide better optical signal response and are more accommodating of angular tolerance variability (relative over or under travel) when the dose setting drum 60 rotates relative to the dose setting member 70.

As shown in FIG. 6, the dose button 72 of the pen injector 1 constitutes the proximal end side of the pen injector 1 and has two concentric grooves 78, 79 formed in its proximal end surface, i.e. the grooves may be formed as depressions or cut-outs in the proximal end face and may extend in the distal direction. These grooves 78, 79 accommodate features of the module 2, allowing the module to be rotated relative to the dose button 72 to find the correct rotational alignment during attachment. The grooves 78, 79 serve as a means by which a set of features are discretely provided on the dose button 72 of the pen that prevent attachment of an incorrect type of module 2, i.e. dedication features, allow the module 2 to be securely attached via clip features 7 and provide access to clutch teeth 61 of the pen mechanism that allow the dose value to be detected/encoded by the module 2. As mentioned above, the features that are used by the module 2 to detect the size of the delivered dose are the clutch teeth 61 that are formed as an equidistantly spaced array of features around the outer circumference at the proximal end of the dose setting drum 60. Although, these encoder features may not need to be clutch teeth 61, but could simply be ‘flags’ for the optical encoder system rotating together with the dose setting drum 60, the arrangement of apertures 77 is useful for permitting access to such encoder features if a module 2 is fitted onto pen 1.

As the module 2 uses a set of optical IR emitters/detectors to encode doses delivered from the pen 1, it is preferred to reduce the level of ‘stray’ IR radiation for example from high ambient light levels if the pen 1 is used outdoors that may interfere with correct performance of the module sensors. The dose setting member 70 components may have features that are intended to reduce/suppress levels of reflected IR radiation. In particular, the tubular sleeve 71 and/or the button 72 may contain an IR absorbing masterbatch and/or may have a spark-eroded textured surface finish.

1-15. (canceled)

16. A drug delivery device for setting and dispensing doses of a liquid drug, the device comprising:
a dose setting and drive mechanism comprising a dose setting member, the dose setting member comprising a distal end and a set of axially directed stop teeth formed at the distal end;
a housing at least partially encasing the dose setting and drive mechanism, the housing comprising a set of counterstop teeth;
wherein the dose setting and drive mechanism is configured to perform a dose setting operation for setting a dose to be delivered by moving the dose setting member relative to the housing, and to perform a dose delivery operation for delivering the set dose by pushing the dose setting member axially towards the housing without relative rotation therebetween;
wherein the set of axially directed stop teeth are configured to engage with the set of counterstop teeth at an end of the dose delivery operation.

17. The drug delivery device of claim 16, wherein the axially directed stop teeth are formed on an outer rim of the dose setting member.

18. The drug delivery device of claim 17, wherein the axially directed stop teeth are formed as portions with increased wall thickness compared to adjacent portions with reduced wall thickness.

19. The drug delivery device of claim 16, wherein the dose setting member comprises two separate components permanently fixed to each other to function as a single component.

20. The drug delivery device according to claim 19, wherein the dose setting member comprises at least one of (i), (ii), (iii) or (iv);
(i) a tubular sleeve comprising the axially directed stop teeth and a ring of clutch teeth extending radially inwards;
(ii) a ring of ramp teeth extending radially inwards;
(iii) a profiling as a gripping surface; or
(iv) a button having a proximal end face and a stem extending distally from the end face, wherein the stem comprises at least one axially extending spline.

21. The drug delivery device of claim 16, wherein the dose setting and drive mechanism comprises a dial clicker that defines discrete positions for the dose setting member with respect to the housing when setting a dose, wherein the dial clicker is formed by a flexible clicker arm and a ring of axially extending splines.

22. The drug delivery device of claim 21, wherein the dose setting and drive mechanism comprises a drive sleeve permanently rotationally fixed to the dose setting member and an inner housing insert permanently rotationally fixed to the housing, wherein the drive sleeve comprises the flexible clicker arm and the ring of axially extending splines is formed on the inner housing insert.

23. The drug delivery device of claim 16, wherein the dose setting and drive mechanism comprises a dose setting drum;
wherein the dose setting and drive mechanism is configured to perform the dose setting operation by rotating the dose setting member relative to the housing, and to perform the dose delivery operation during rotation of the dose setting drum relative to at least one of the dose setting member or the housing;
wherein a clutch acts between the dose setting member and the dose setting drum; and
wherein the clutch is engaged during the dose setting operation so that the dose setting member and dose setting drum rotate together, and is disengaged during the dose delivery operation to allow relative rotational movement between the dose setting drum and the dose setting member.

24. The drug delivery device of claim 23, wherein the dose setting drum comprises encoder features formed in a circular pattern for detecting rotational movement of the dose setting drum relative to the dose setting member or the housing.

25. The drug delivery device of claim 24, wherein the encoder features are proximally facing clutch teeth provided on the dose setting drum.

26. The drug delivery device of claim 24, wherein the dose setting member has at least one aperture located in a proximal end surface that is coincident with at least a portion of the clutch teeth of the dose setting drum.

27. The drug delivery device of claim 26, wherein the at least one aperture is located at the proximal end of a groove.

28. The drug delivery device of claim 27, wherein the groove is disposed at the distal end of an outer groove of two concentrical grooves formed in the proximal end surface of the dose setting member.

29. The drug delivery device of claim 16, wherein the dose setting member is provided with attachment features for attaching a separate module.

30. A dose recording system comprising:
a drug delivery device comprising a dose setting and drive mechanism comprising a dose setting member; and
an electronic module for attachment to the dose setting member of the drug delivery device, the electronic module comprising:
at least one sensor;
a processor configured to control operation of the at least one sensor and to at least one of process or store signals from the at least one sensor; and
attachment features for attaching the electronic module to the dose setting member.

31. The dose recording system of claim 30, wherein the electronic module comprises a cap for receiving a proximal end of the dose setting member.

32. The dose recording system of claim 31, wherein the dose setting and drive mechanism of the drug delivery device comprises a dose setting drum having clutch teeth;
wherein the at least one sensor of the electronic module comprises at least one set of optical IR emitters/detectors located coincident with at least a portion of the clutch teeth of the dose setting drum when the electronic module is attached to the dose setting member.

33. The dose recording system of claim 30, wherein the electronic module comprises a chassis holding the at least one sensor and the processor.

34. The dose recording system of claim 33, wherein the dose setting member has at least one aperture located in a proximal end surface that is coincident with at least a portion of the clutch teeth of the dose setting drum; and
wherein the chassis comprises at least one light pipe extending through the at least one aperture in the proximal end surface of the dose setting member.

35. The dost recording system of claim 30, wherein the dose setting member comprises a distal end and a set of axially directed stop teeth formed at the distal end;
wherein the drug delivery device comprises a housing at least partially encasing the dose setting and drive mechanism, the housing comprising a set of counterstop teeth;
wherein the dose setting and drive mechanism is configured to perform a dose setting operation for setting a dose to be delivered by moving the dose setting member relative to the housing, and a dose delivery operation for delivering the dose by pushing the dose setting member axially towards the housing without relative rotation therebetween; and
wherein the set of axially directed stop teeth are configured to engage with the set of counterstop teeth at an end of the dose delivery operation.
